(19) United States
(12) Patent Application Publication
(10) Pub. No.: US 2025/0073386 A1
(43) Pub. Date: Mar. 6, 2025

(54) PRESSURE SENSOR FOR A DEVICE FOR DELIVERING INSULIN TO A USER

(71) Applicant: AITA BIO INC., New York, NY (US)

(72) Inventors: Dilan Casanovas Mack, New York, NY (US); Jacob Menture, New York, NY (US)

(21) Appl. No.: 18/724,106

(22) PCT Filed: Jan. 16, 2023
(86) PCT No.: PCT/US2023/010875

§ 371 Date: Jun. 25, 2024

Related U.S. Application Data
(60) Provisional application No. 63/300,598, filed on Jan. 18, 2022.

(51) Int. Cl. A61M 5/142

(52) U.S. Cl. CPC A61M 5/14248; A61M 2005/14252; A61M 2205/0294; A61M 2205/3344; A61M 2230/201

(57) ABSTRACT

A device for delivering insulin to a user, the device configured as a wearable apparatus or system in which continuous glucose monitoring (CGM), insulin delivery and control functionality are provided to ensure insulin is delivered to the user, the device comprising: a reservoir for storing insulin; a catheter configured to deliver insulin to a subcutaneous layer of the user; an introducer needle movable with respect to the catheter, the introducer needle configured to facilitate (a) insertion of the catheter into the subcutaneous layer of the user and (b) removal of the introducer needle to enable delivery of insulin through the catheter; a micropump, in fluid communication with the reservoir and catheter, for pumping insulin from the reservoir through the catheter; and a pressure sensor separate from and in fluid communication with an outlet port of the micropump, the sensor comprising: a first wafer defining an inlet port, and outlet port and fluid channel communicating with the inlet and outlet ports; a second wafer covering the fluid channel and including a pressure sensing mechanism; and a piezoelectric device layered on the first wafer and configured to generate a signal in response to the pressure sensing mechanism that is representative of pressure within channel.

### CROSS-REFERENCE TO RELATED APPLICATIONS

This application claims priority to the U.S. provisional application No. 63/300,598, filed Jan. 18, 2022, entitled “Pressure Sensor For Wearable Apparatus for Diabetes Management”, which is incorporated by reference herein.

### FIELD OF THE INVENTION

The present invention relates to a pressure sensor for a device for delivering insulin to a user, the device being a wearable apparatus of an infusion system for diabetes management.

### BACKGROUND OF THE INVENTION

Insulin pumps help people with diabetes to conveniently manage their blood sugar. These devices deliver insulin at specific times. Insulin patch pumps or pods are one type of insulin pump. The pods are wearable devices that adhere to the skin of a user using an adhesive patch. The pods deliver insulin from a chamber and internal cannula based on separately acquired CGM sensor readings. The pods are controlled wirelessly with a handheld controller. Sensors are crucial to the safety of the user.

It would be advantageous to provide improvements to insulin pumps described above.

### SUMMARY OF THE INVENTION

A pressure sensor is disclosed for a device for delivering insulin to a user, the device being wearable apparatus of an infusion system for diabetes management.

In accordance with an embodiment of the present disclosure, A device for delivering insulin to a user, the device configured as a wearable apparatus or system in which continuous glucose monitoring (CGM), insulin delivery and control functionality are provided to ensure insulin is delivered to the user, the device comprising: a reservoir for storing insulin; a catheter configured to deliver insulin to a subcutaneous layer of the user; an introducer needle movable with respect to the catheter, the introducer needle configured to facilitate (a) insertion of the catheter into the subcutaneous layer of the user and (b) removal of the introducer needle to enable delivery of insulin through the catheter; a micropump, in fluid communication with the reservoir and catheter, for pumping insulin from the reservoir through the catheter; and a pressure sensor separate from and in fluid communication with an outlet port of the micropump, the sensor comprising: a first wafer defining an inlet port, and outlet port and fluid channel communicating with the inlet and outlet ports; a second wafer covering the fluid channel and including a pressure sensing mechanism; and a piezoelectric device layered on the second wafer and configured to generate a signal in response to the pressure sensing mechanism that is representative of pressure within channel.

In accordance with another embodiment of the disclosure, a device for delivering insulin to a user, the device configured as a wearable apparatus or system in which continuous glucose monitoring (CGM), insulin delivery and control functionality are provided to ensure insulin is delivered to the user, the device comprising: a reservoir for storing insulin; a catheter configured to deliver insulin to a subcutaneous layer of the user; a micropump, in fluid communication with the reservoir and catheter, for pumping insulin from the reservoir through the catheter; and a pressure sensor separate from and in fluid communication with an outlet port of the micropump, the sensor comprising: first wafer and second wafers defining an inlet port, and outlet port and fluid channel communicating with the inlet and outlet ports; the second wafer including a pressure sensing mechanism; and a piezoelectric device layered on the second wafer and configured to generate a signal in response to the pressure sensing mechanism that is representative of pressure from the insulin within channel.

In accordance with another embodiment, a device for delivering medicament to a user, the device comprising: a reservoir for storing the medicament; a catheter configured to deliver medicament to the user; an introducer needle movable with respect to the catheter, the introducer needle configured to facilitate (a) insertion of the catheter into the user and (b) removal of the introducer needle to enable delivery of medicament through the catheter; a micropump, in fluid communication with the reservoir and the catheter, for pumping the medicament from the reservoir through the catheter; and a pressure sensor separate from and in fluid communication with an outlet port of the micropump, the sensor comprising: a first wafer defining an inlet port, and outlet port and fluid channel communicating with the inlet and outlet ports; a second wafer covering the fluid channel and including a pressure sensing mechanism; and a piezoelectric device layered on the second wafer and configured to generate a signal in response to the pressure sensing mechanism that is representative of pressure within channel.

In accordance with another embodiment, a device for delivering insulin to a user, the device configured as a wearable apparatus or system in which continuous glucose monitoring (CGM), insulin delivery and control functionality are provided to ensure insulin is delivered to the user, the device comprising: a reservoir for storing insulin; a catheter configured to deliver insulin to a subcutaneous layer of the user; a micropump, in fluid communication with the reservoir and catheter, for pumping insulin from the reservoir through the catheter; and a pressure sensor separate from and in fluid communication with an outlet port of the micropump, the sensor comprising: first wafer and second wafers defining an inlet port, and outlet port and fluid channel communicating with the inlet and outlet ports; the second wafer including a pressure sensing mechanism; and a piezoelectric device layered on the second wafer and configured to generate a signal in response to the pressure sensing mechanism that is representative of pressure from the insulin within channel.

### BRIEF DESCRIPTION OF DRAWINGS

FIG. 1 depicts a perspective view of an example pressure sensor for a device for delivering insulin, the device configured as a wearable apparatus or system that is part of an infusion system for diabetes management.

FIG. 2 depicts a block diagram of several components of the device including the pressure sensor shown in FIG. 1 in fluidic communication.

FIG. 3 depicts an exploded view of a two-wafer (top and bottom) structure of the example pressure sensor shown in FIG. 1.

FIG. 4 depicts a plan view of the bottom wafer of the example pressure sensor shown in FIG. 1.

FIG. 5 depicts a cross-sectional view of the example pressure sensor shown in FIG. 1.

FIG. 6 depicts a cross-sectional view of the example pressure sensor shown in FIG. 1 including compressed tubing.

### DETAILED DESCRIPTION OF THE INVENTION

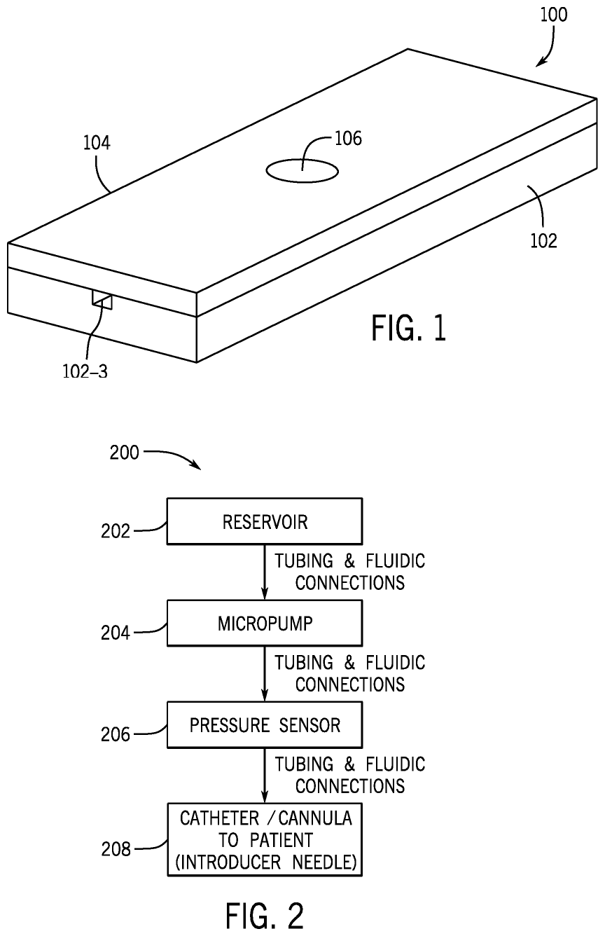

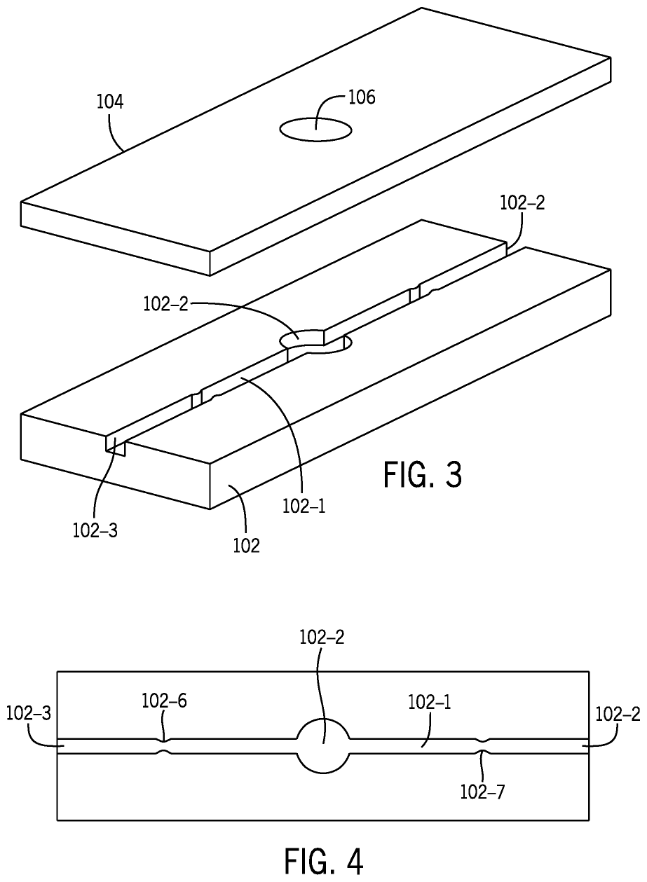

FIG. 1 depicts a perspective view of example pressure sensor 100 and FIGS. 3-6 depict various other views of pressure sensor 100. Sensor 100 is a component of device 200 for delivering insulin or other fluid medicament such as small molecule pharmaceutical solutions, large molecule or protein drug solutions, saline solutions, blood or other fluids known to those skilled in the art. Device 200 is configured as a wearable apparatus or system (that is part of an infusion system for diabetes management) in which continuous glucose monitoring (CGM), insulin delivery and control functionality are provided to ensure insulin is delivered at very precise rates and has the capability of detecting occlusions in real time. Pressure sensor 100 is integrated into the fluidic pathway in device 200 as shown in the block diagram of FIG. 2 (as pressure sensor 206). Pressure sensor 100 functions as a patient/user safety feature of device 200 to continually monitor pressure changes in the fluidic pathway as described in more detail below.

As shown in FIG. 2, device 200 incorporates several components or modules (not shown) in the fluidic pathway including reservoir 202 for storing the insulin, micropump 204 for pumping insulin, pressure sensor 206 (sensor 100 renumbered in FIG. 2 only) and insulin cannula or catheter 208. Catheter 208 is inserted via an introducer needle (shown in box 208) as known to those skilled in the art.

Pressure sensor 100 (sensor 206 in FIG. 2) is configured as a separate component in device 200 that is not intended to be part of or integrated on a printed circuit board or micropump 204 itself. Pressure sensor 100 is a standalone pressure sensor (chip) that integrates with, i.e., is in fluid communication with an outlet port of the micropump 204 as described in detail below. Device 200 also includes an insulin catheter or needle, a continuous glucose monitoring (CGM) sensor wire, control circuitry-integrated circuit (IC) and a battery for powering the IC, battery and power controller, microcontroller unit and other sensors (these components are not shown).

Micropump 204 is a MEMS (micro-electro-mechanical systems) device or pump, as known to those skilled in the art, that can be used for pumping fluid, as valves used for regulating flow, actuators used for moving or controlling the micropump and/or sensors used for sensing pressure and/or flow. The MEMS micropump is a two wafer structure wherein one or more chambers are sandwiched between or defined by the two wafers as known to those skilled in the art. The MEMS device incorporates one or more piezoelectric devices that function as piezoelectric actuators or elements for pumping fluid, piezoelectric valves for preventing fluid flow and/or a sensor for sensing pressure or flow through the chambers of micropump 204. Example piezoelectric devices includes (1) piezoelectrical devices, transducers (PZT), and (2) sensors and piezoresistive transducers and sensors, However, piezoelectric devices may be described hereinafter with respect to FIGS. 1-6 as the piezo device. Other MEMS or non-MEMS structures or technology may also be used as micropump 204 to achieve desired results as known to those skilled in the art. Micropump 204 may be used in a drug infusion system for infusing insulin or other fluidic medication to a patient (user). Medication may include small molecule pharmaceutical solutions, large molecule or protein drug solutions, saline solutions, blood or other fluids known to those skilled in the art.

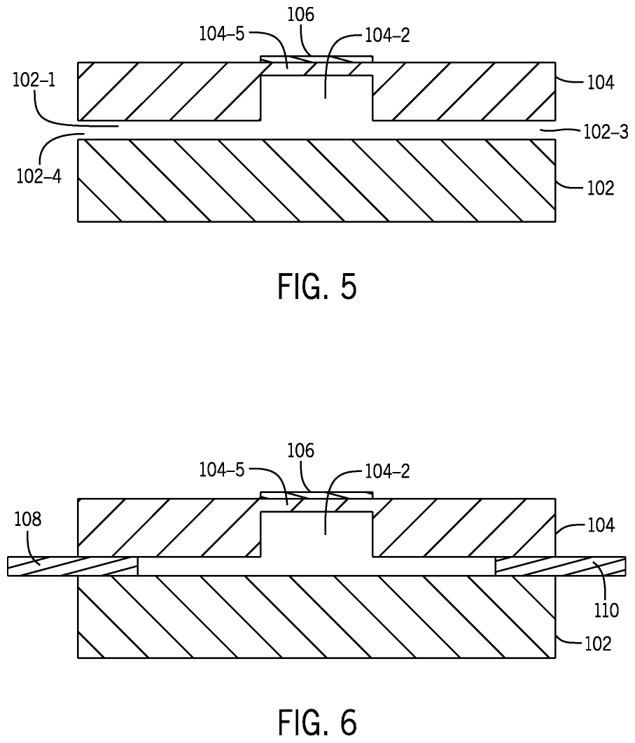

As seen in FIG. 1 and FIGS. 3-6, pressure sensor 100 is configured as a two-wafer structure, wherein bottom wafer 102 is etched to create microfluidic channel 102-1, circular section or opening 102-2 within the channel and inlet and outlet ports 102-3, 102-4 and top wafer 104 is configured to seal fluidic channel 102-1 created on bottom wafer 102-1. Additionally, although not shown in FIGS. 5 and 6, in the same location as the bottom opening, top wafer 104 is etched in the same shape and dimension to create thin (silicon) section or membrane 102-5 that functions as a pressure sensing mechanism of sensor 100. The membrane can have a thickness of 8-10 um, but those skilled in the art know that other thickness may be used to achieve desired results. Sensor 100 also includes a piezoelectric device or element 106 (e.g., piezoelectric or piezoresistive transducer) as described above with respect to micropump 204 that is layered on top of membrane 102-5 of wafer 104. In operation, membrane 102-5 will deflect in response to pressure within fluid channel 102-1. In response, piezoelectric device 106 generates a voltage as known to those skilled in the art representative of this fluid pressure.

Bottom wafer 102 incorporates indentations 102-6, 102-7 in fluidic channel 102-1 to facilitate press fitting of tubing 108,110. As seen in FIG. 6, fluidic connection between micropump 104 and cannula or catheter 208 is achieved using tubing 108,110. In some detail, a section of tubing 108,110 (e.g., plastic or rubber tubing) can be inserted and press fitted through both inlet and outlet ports 102-3, 102-4, respectively and into channel 102-1 to create this fluidic connection.

As indicated above, pressure sensor 100 is a standalone/independent component to be integrated into the fluidic path of device 200 that is configured as a wearable apparatus or system for diabetes management. The pressure sensor structure is integrated in device 200 but separately from micropump 204 and other components in the fluidic pathway. Microfluidic channel 102-1 is preferably between 100-700 um wide that starts off an as inlet port or opening. Down the fluidic path, towards the middle of the sensor 100 (chip), there is a circular opening with a diameter of 0.5-3 mm which will be the position of pressure sensing mechanism (wall 104-5 also as membrane 104-5 as described below). The opening can be circular, or any polygonal shape. The top of the opening will have the wall 104-5 as membrane 104-5 with a preferable thickness of 8-50 um which will function (together with piezoelectric transducer 106) as the pressure sensing mechanism. (The thin section of wafer 104 includes the wall 104-5). As the pressure in the fluidic path increases, the membrane will deflect according to that specific pressure in the fluidic path. After the opening, the sensor chip will go back to a microfluidic channel that is preferably 100-700 um wide and it ends at outlet port 102-2. The channels can be rectangular, square or circular. The measurements described hereinabove are preferred values but those skilled in the art know that other measurements and/or dimensions may be used to achieve desired results.

As indicated above, pressure sensor 100 is a standalone/independent component to be integrated into the fluidic path of device 200 for delivering insulin that is configured as a wearable apparatus or system for diabetes management. However, the standalone component may alternatively incorporate two (or more) of the same or different piezoelectric devices on the same chip or multiple chips using the same fluidic path. The two piezoelectric devices can be placed at each end of the chip. There are a few benefits of adding two of these on the same chip. First, if the piezo membranes have the same diameter, the difference in pressure signals can be used to derive the flow rate at which the micropump is pumping insulin.

Second, if the piezo membranes have two different diameters, the combination may sense (generate) two different pressure ranges. This is important because there are two different delivery rates for insulin delivery: basal and bolus. The basal delivery is a much slower delivery rate and a bolus is at a much higher rate. Therefore, the basal delivery rate will generate a much lower pressure than the bolus delivery rate. The larger piezo membrane can be used to monitor the pressure during basal delivery rate because it will have a lower pressure range while the smaller transducer could be used to monitor the pressure during the bolus delivery.

It is to be understood that the disclosure teaches examples of the illustrative embodiments and that many variations of the invention can easily be devised by those skilled in the art after reading this disclosure and that the scope of the present invention is to be determined by the claims below.

What is claimed is:

1. A device for delivering insulin to a user, the device configured as a wearable apparatus or system in which continuous glucose monitoring (CGM), insulin delivery and control functionality are provided to ensure insulin is delivered to the user, the device comprising:
a reservoir for storing insulin;
a catheter configured to deliver insulin to a subcutaneous layer of the user;
an introducer needle movable with respect to the catheter, the introducer needle configured to facilitate (a) insertion of the catheter into the subcutaneous layer of the user and (b) removal of the introducer needle to enable delivery of insulin through the catheter;
a micropump, in fluid communication with the reservoir and the catheter, for pumping insulin from the reservoir through the catheter; and
a pressure sensor separate from and in fluid communication with an outlet port of the micropump, the sensor comprising:
a first wafer defining an inlet port, and outlet port and fluid channel communicating with the inlet and outlet ports;
a second wafer covering the fluid channel and including a pressure sensing mechanism; and
a piezoelectric device layered on the second wafer and configured to generate a signal in response to the pressure sensing mechanism that is representative of pressure within channel.

2. The device of claim 1 wherein the channel includes a circular section.

3. The device of claim 2 wherein the pressure sensing mechanism is a thin section of the second wafer that corresponds in location with the circular section, the thin section includes a wall, that functions as a membrane, that communicates with the fluid channel.

4. The device of claim 2 wherein the piezoelectric device is layered over the thin section.

5. The device of claim 1 wherein first wafer includes indentations in the fluid channel to facilitate press fitting of tubing.

6. A device for delivering insulin to a user, the device configured as a wearable apparatus or system in which continuous glucose monitoring (CGM), insulin delivery and control functionality are provided to ensure insulin is delivered to the user, the device comprising:
a reservoir for storing insulin;
a catheter configured to deliver insulin to a subcutaneous layer of the user;
a micropump, in fluid communication with the reservoir and catheter, for pumping insulin from the reservoir through the catheter; and
a pressure sensor separate from and in fluid communication with an outlet port of the micropump, the sensor comprising:
first wafer and second wafers defining an inlet port, and outlet port and fluid channel communicating with the inlet and outlet ports;
the second wafer including a pressure sensing mechanism; and
a piezoelectric device layered on the second wafer and configured to generate a signal in response to the pressure sensing mechanism that is representative of pressure from the insulin within channel.

7. The device of claim 6 wherein the wafer comprises the channel and an opening to the channel and the second wafer covers the opening in channel, thereby functioning as a wall to channel.

8. The device of claim 6 wherein the channel includes a circular section.

9. The device of claim 8 wherein the pressure sensing mechanism is a thin section of the second wafer that corresponds in location with the circular section, the thin section includes a wall, that functions as a membrane, that communicates with the fluid within the fluid channel.

10. The device of claim 9 wherein the piezoelectric device is layered over the thin section.

11. The device of claim 6 wherein the first wafer includes indentations in the fluid channel to facilitate press fitting of tubing.

12. The device of claim 6 wherein the piezoelectric device is a piezoelectric or piezoresistive transducer.

13. A device for delivering medicament to a user, the device comprising:
a reservoir for storing the medicament;
a catheter configured to deliver medicament to the user;
an introducer needle movable with respect to the catheter, the introducer needle configured to facilitate (a) insertion of the catheter into the user and (b) removal of the introducer needle to enable delivery of medicament through the catheter;
a micropump, in fluid communication with the reservoir and the catheter, for pumping the medicament from the reservoir through the catheter; and
a pressure sensor separate from and in fluid communication with an outlet port of the micropump, the sensor comprising:
a first wafer defining an inlet port, and outlet port and fluid channel communicating with the inlet and outlet ports;
a second wafer covering the fluid channel and including a pressure sensing mechanism; and
a piezoelectric device layered on the second wafer and configured to generate a signal in response to the pressure sensing mechanism that is representative of pressure within channel.

14. The device of claim 13 wherein the channel includes a circular section.

15. The device of claim 14 wherein the pressure sensing mechanism is a thin section of the second wafer that corresponds in location with the circular section, the thin section includes a wall, that functions as a membrane, that communicates with the fluid channel.

16. The device of claim 15 wherein the piezoelectric device is layered over the thin section.

17. The device of claim 13 wherein first wafer includes indentations in the fluid channel to facilitate press fitting of tubing.

18. A device for delivering insulin to a user, the device configured as a wearable apparatus or system in which continuous glucose monitoring (CGM), insulin delivery and control functionality are provided to ensure insulin is delivered to the user, the device comprising:
a reservoir for storing insulin;
a catheter configured to deliver insulin to a subcutaneous layer of the user;
a micropump, in fluid communication with the reservoir and catheter, for pumping insulin from the reservoir through the catheter; and
a pressure sensor separate from and in fluid communication with an outlet port of the micropump, the sensor comprising:
first wafer and second wafers defining an inlet port, and outlet port and fluid channel communicating with the inlet and outlet ports;
the second wafer including a pressure sensing mechanism; and
a piezoelectric device layered on the second wafer and configured to generate a signal in response to the pressure sensing mechanism that is representative of pressure from the insulin within channel.

19. The device of claim 18 wherein the wafer comprises the channel and an opening to the channel and the second wafer covers the opening in channel, thereby functioning as a wall to channel.

20. The device of claim 18 wherein the channel includes a circular section.

21. The device of claim 20 wherein the pressure sensing mechanism is a thin section of the second wafer that corresponds in location with the circular section, the thin section includes wall, that functions as a membrane, that communicates with the fluid within the fluid channel.

22. The device of claim 21 wherein the piezoelectric device is layered over the thin section.

23. The device of claim 18 wherein first wafer includes indentations in the fluid channel to facilitate press fitting of tubing.

24. The device of claim 18 wherein the piezoelectric device is a piezoelectric or piezoresistive transducer.
